(19) United States
(12) Patent Application Publication
(10) Pub. No.: US 2026/0104374 A1
(43) Pub. Date: Apr. 16, 2026

(54) SEMICONDUCTOR DEVICE IMAGING METHOD

(71) Applicant: Samsung Electronics Co., Ltd., Suwon-si (KR)

(72) Inventors: Taejin KWON, Suwon-si (KR); Jeonghoi KIM, Suwon-si (KR); Jongcheon SUN, Suwon-si (KR); Su-Young LEE, Suwon-si (KR); Hyeongcheol LEE, Suwon-si (KR)

(73) Assignee: Samsung Electronics Co., Ltd., Suwon-si (KR)

(21) Appl. No.: 19/213,350

(22) Filed: May 20, 2025

(30) Foreign Application Priority Data
Oct. 14, 2024 (KR) 10-2024-0139726

(51) Int. Cl. G01N 23/2252; G01N 23/04; G01N 23/083; G01N 33/00

(52) U.S. Cl. CPC G01N 23/2252; G01N 23/04; G01N 23/083; G01N 33/0095; G01N 2223/04; G01N 2223/079; G01N 2223/3301; G01N 2223/3306

(57) ABSTRACT

A method of imaging a semiconductor device includes forming an X-ray source layer on a first surface of a semiconductor substrate, mounting the semiconductor substrate in a semiconductor device imaging apparatus, the semiconductor device imaging apparatus including an electron-beam feeder and an X-ray detector, irradiating electron-beams on the X-ray source layer of the semiconductor substrate using the electron-beam feeder, and measuring X-rays emitted from the X-ray source layer and having passed through the semiconductor substrate using the X-ray detector.

### CROSS-REFERENCE TO RELATED APPLICATION

This U.S. non-provisional application claims priority under 35 U.S.C. § 119 to Korean Patent Application No. 10-2024-0139726, filed on Oct. 14, 2024, in the Korean Intellectual Property Office, the disclosure of which is incorporated by reference herein in its entirety.

### BACKGROUND

Example embodiments are directed to a method of imaging a semiconductor device using X-rays.

As performance of modern electronic devices improves, semiconductor devices used in the electronic devices become increasingly miniaturized. However, as semiconductor devices become smaller and smaller, it may be challenging to fabricate the semiconductor devices. Conventional inspection methods may be limited in inspecting the relatively smaller, fine structure of the semiconductor devices, and the likelihood of malfunctions or defects in the semiconductor devices being undetected may increase.

Accordingly, various inspection and diagnosis technologies are being studied to improve the quality and reliability of semiconductor devices, and it is desirable to improve device performance and productivity.

### SUMMARY

Example embodiments of the inventive concepts provide a method of imaging a semiconductor device by obtaining a relatively higher resolution X-ray image of the semiconductor device.

The solutions provided by example embodiments are not limited to the above-mentioned solutions, and other solutions not mentioned may be clearly understood by those of ordinary skill in the art from the following description.

According to some example embodiments of the inventive concepts, a method of imaging a semiconductor device includes forming an X-ray source layer on a first surface of a semiconductor substrate, mounting the semiconductor substrate in a semiconductor device imaging apparatus, the semiconductor device imaging apparatus including an electron-beam feeder and an X-ray detector, irradiating electron-beams on the X-ray source layer of the semiconductor substrate using the electron-beam feeder, and measuring X-rays emitted from the X-ray source layer and having passed through the semiconductor substrate using the X-ray detector.

According to some example embodiments of the inventive concepts, a method of imaging a semiconductor device includes mounting a semiconductor substrate in a semiconductor device imaging apparatus including an electron-beam feeder and an X-ray detector, irradiating electron-beams on an X-ray source layer of the semiconductor substrate using the electron-beam feeder, and measuring X-rays emitted from the X-ray source layer and having passed through the semiconductor substrate using the X-ray detector. The semiconductor substrate includes the X-ray source layer on a first surface of the semiconductor substrate, and the semiconductor substrate is mounted in the semiconductor device imaging apparatus such that the X-ray source layer faces the electron-beam feeder.

According to some example embodiments of the inventive concepts, a method of imaging a semiconductor device includes forming a dummy layer and an X-ray source layer on a first surface of the semiconductor substrate to be measured, the dummy layer and the X-ray source layer covering an entirety of the first surface, mounting the semiconductor substrate between an electron-beam feeder and an X-ray detector of a semiconductor device imaging apparatus, the semiconductor substrate being mounted such that the X-ray source layer faces the electron-beam feeder, and the electron-beam feeder and the X-ray detector being spaced apart in a first direction from each other, rotating the semiconductor substrate about a rotational axis passing through a center of the first surface of the semiconductor substrate and extending in a direction of a line that is normal to the first surface of the semiconductor substrate, irradiating electron-beams toward the X-ray source layer of the semiconductor substrate using the electron-beam feeder, directly striking the X-ray source layer with the electron-beams to cause the X-ray source layer to form X-rays, and detecting the X-rays that pass through the semiconductor substrate using the X-ray detector. The semiconductor substrate is mounted in the semiconductor device imaging apparatus such that the direction of the line normal to the first surface of the semiconductor substrate and the first direction intersect each other.

According to some example embodiments, a semiconductor device imaging system includes a semiconductor substrate having a first surface and an X-ray source layer on the first surface, and a semiconductor device imaging apparatus. The semiconductor device imaging apparatus includes an electron-beam feeder configured to irradiate electron-beams on the X-ray source layer of the semiconductor substrate, and an X-ray detector configured to measure X-rays emitted from the X-ray source layer and having passed through the semiconductor substrate.

According to some example embodiments, the semiconductor device imaging system may further include an anode configured to accelerate the electron-beams towards the semiconductor substrate based on a voltage applied between the electron-beam feeder and the anode, a magnetic lens configured to focus and accelerate the electron-beams towards the semiconductor substrate, a scanning coil configured to scan the electron-beams EB on the semiconductor substrate, and an objective lens positioned between the semiconductor substrate and the scanning coil and configured to focus the electron-beams deflected by the scanning coil on the X-ray source layer of the semiconductor substrate.

According to some example embodiments, the electron-beam feeder and the X-ray detector are spaced apart in a first direction, and the semiconductor substrate is mounted in the semiconductor device imaging apparatus such that the first surface of the semiconductor substrate faces the electron-beam feeder and a second surface of the semiconductor substrate faces the X-ray detector, the second surface being opposite to the first surface.

According to some example embodiments, the semiconductor device imaging apparatus further includes an electron-beam detector configured to measure electron-beams reflected from at least one of the X-ray source layer or the semiconductor substrate among the irradiated electron-beams.

According to some example embodiments, semiconductor substrate is mounted in the semiconductor device imaging apparatus such that a line that is normal to the first surface of the semiconductor substrate extends in a second direction, and wherein the second direction is parallel to a first direction including the electron-beam feeder and the X-ray detector.

According to some example embodiments, the semiconductor substrate is mounted in the semiconductor device imaging apparatus such that a line that is normal to the first surface of the semiconductor substrate extends in a second direction, and wherein the second direction intersects a first direction including the electron-beam feeder and the X-ray detector.

### BRIEF DESCRIPTION OF THE DRAWINGS

Example embodiments will be more clearly understood from the following detailed description taken in conjunction with the accompanying drawings in which:

FIG. 1 is a flowchart schematically illustrating a method of imaging a semiconductor device, according to some example embodiments.

FIG. 2 is a diagram schematically illustrating a semiconductor device imaging apparatus and a semiconductor substrate, according to some example embodiments.

FIG. 3 is an enlarged view of a portion of the semiconductor substrate of FIG. 2.

FIG. 4 is a flowchart schematically illustrating a method of imaging a semiconductor device, according to some example embodiments.

FIG. 5 is a diagram schematically illustrating a semiconductor device imaging apparatus and a semiconductor substrate, according to some example embodiments.

FIG. 6 is an enlarged view of a portion of the semiconductor substrate of FIG. 5.

FIG. 7 is a flowchart schematically illustrating a method of imaging a semiconductor device, according to some example embodiments.

FIG. 8 is a diagram schematically illustrating a semiconductor device imaging apparatus and a semiconductor substrate, according to some example embodiments.

FIG. 9 is a flowchart schematically illustrating a method of imaging a semiconductor device, according to some example embodiments.

FIG. 10 is a diagram schematically illustrating a semiconductor device imaging apparatus and a semiconductor substrate, according to some example embodiments.

FIG. 11 is a block diagram illustrating an example computer system with which the methods and operations illustrated in FIGS. 1, 4, 7, and 9, and other tasks described herein can be implemented, according to some example embodiments.

### DETAILED DESCRIPTION OF EXAMPLE EMBODIMENTS

Since the present embodiments may undergo various changes and have various forms, some example embodiments will be illustrated in the drawings and described in detail. However, this is not intended to limit the present embodiments to a specific form of disclosure.

As used herein, the term “and/or” includes any and all combinations of one or more of the associated listed items. Expressions such as “at least one of,” when preceding a list of elements, modify the entire list of elements and do not modify the individual elements of the list. For example, “at least one of A, B, and C,” and similar language (e.g., “at least one selected from the group consisting of A, B, and C,” “at least one of A, B, or C”) may be construed as A only, B only, C only, or any combination of two or more of A, B, and C, such as, for instance, ABC, AB, BC, and AC.

It will be understood that elements and/or properties thereof (e.g., structures, surfaces, directions, or the like), which may be referred to as being “perpendicular,” “parallel,” “coplanar,” or the like with regard to other elements and/or properties thereof (e.g., structures, surfaces, directions, or the like) may be “perpendicular,” “parallel,” “coplanar,” or the like or may be “substantially perpendicular,” “substantially parallel,” “substantially coplanar,” respectively, with regard to the other elements and/or properties thereof.

It will be understood that elements and/or properties thereof may be recited herein as being “the same” or “equal” as other elements, and it will be further understood that elements and/or properties thereof recited herein as being “identical” to, “the same” as, or “equal” to other elements may be “identical” to, “the same” as, or “equal” to or “substantially identical” to, “substantially the same” as or “substantially equal” to the other elements and/or properties thereof. Elements and/or properties thereof that are “substantially identical” to, “substantially the same” as or “substantially equal” to other elements and/or properties thereof will be understood to include elements and/or properties thereof that are identical to, the same as, or equal to the other elements and/or properties thereof within manufacturing tolerances and/or material tolerances. Elements and/or properties thereof that are identical or substantially identical to and/or the same or substantially the same as other elements and/or properties thereof may be structurally the same or substantially the same, functionally the same or substantially the same, and/or compositionally the same or substantially the same. While the term “same,” “equal” or “identical” may be used in description of some example embodiments, it should be understood that some imprecisions may exist. Thus, when one element, value, and/or property is referred to as being the same as another element, value, and/or property, it should be understood that an element, value, and/or property is the same as another element, value, and/or property within a desired manufacturing or operational tolerance range (e.g., +10%).

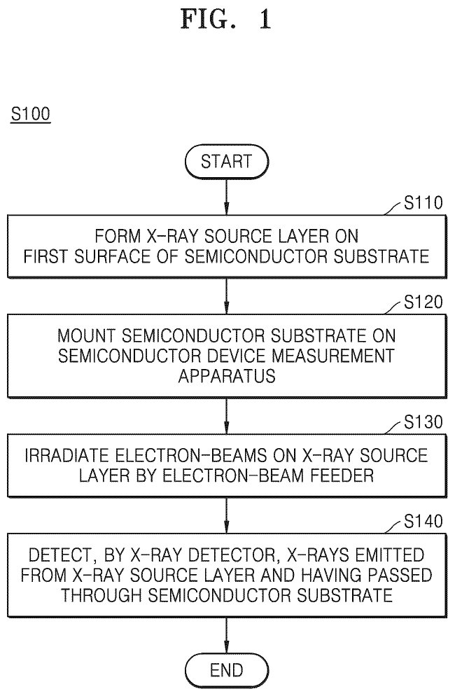

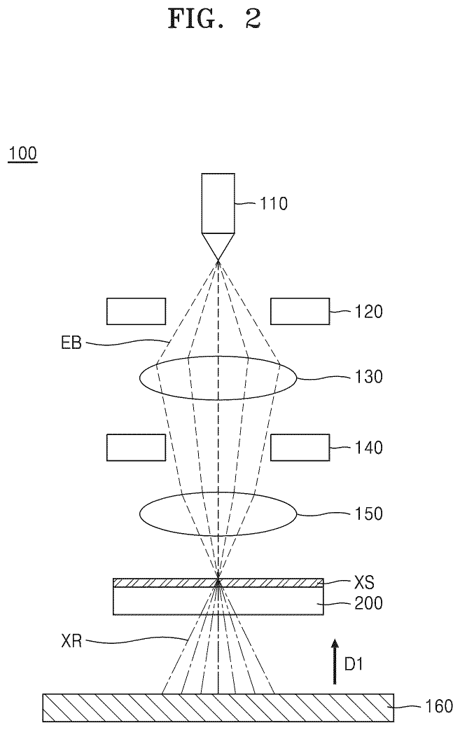

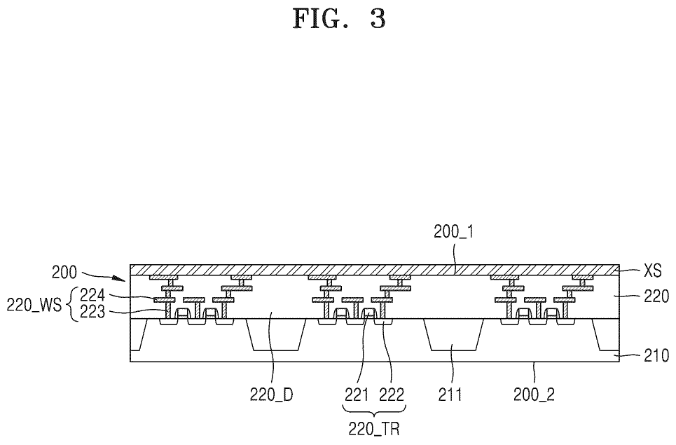

FIG. 1 is a flowchart schematically illustrating a method of imaging a semiconductor device (S100) according to some example embodiments. FIG. 2 is a diagram schematically illustrating a semiconductor device imaging apparatus 100 and a semiconductor substrate 200 according to some example embodiments. FIG. 3 is an enlarged view of a portion of the semiconductor substrate 200 of FIG. 2. It is understood that additional operations can be provided before, during, and after the operations in FIG. 1, and some of the operations described below can be replaced or eliminated, for additional embodiments of the method. The order of the operations/processes may be interchangeable, or two or more operations can be performed simultaneously.

Referring to FIGS. 1 to 3, a semiconductor device imaging method S100 may include an operation S110 of forming an X-ray source layer XS on a first surface 200_1 of a semiconductor substrate 200, an operation S120 of mounting the semiconductor substrate 200 on a semiconductor device imaging apparatus 100, an operation S130 of irradiating electron-beams EB on the X-ray source layer XS using an electron-beam feeder 110, and an operation S140 of detecting, by an X-ray detector 160, X-rays XR emitted from the X-ray source layer XS and having passed through the semiconductor substrate 200.

The semiconductor substrate 200 may be described with reference to FIG. 3.

The semiconductor substrate 200 may include a first surface 200_1 and a second surface 200_2 opposite to the first surface 200_1. The semiconductor substrate 200 may include a base layer 210 and an active layer 220. In some example embodiments, the surface on which the active layer 220 is formed may be referred to as the first surface 200_1 of the semiconductor substrate 200, and the surface on which the base layer 210 is located may be referred to as the second surface 200_2 of the semiconductor substrate 200. Referring to FIG. 3, a top surface of the semiconductor substrate 200 may be a first surface 200_1 of the semiconductor substrate 200, and a bottom surface of the semiconductor substrate 200 may be a second surface 200_2 of the semiconductor substrate 200.

The active layer 220 including a plurality of semiconductor devices 220_TR may be formed on one surface of the base layer 210. In the base layer 210, an active region may be defined by device isolation layers 211, and the plurality of semiconductor devices 220_TR may be formed on the active region.

The base layer 210 may include a semiconductor material, for example, a group IV semiconductor, a group III-V compound semiconductor, or a group II-VI oxide semiconductor. For example, the group IV semiconductor may include silicon (Si), germanium (Ge), or silicon-germanium (SiGe). The base layer 210 may be provided as a bulk wafer or an epitaxial layer. In some example embodiments, the base layer 210 may include a silicon-on-insulator (SOI) substrate or a germanium-on-insulator (GeOI) substrate.

The active layer 220 may include the plurality of semiconductor devices 220_TR, a wiring structure 220_WS, and an insulating layer 220_D surrounding the plurality of semiconductor devices 220_TR and the wiring structure 220_WS.

In some example embodiments, each of the plurality of semiconductor devices 220_TR may include a gate 221 and source/drain regions 222 arranged on portions of the base layer 210 on both sides of the gate 221. The plurality of semiconductor devices 220_TR of the active layer 220 may include various microelectronic devices, for example, metal-oxide-semiconductor field effect transistors (MOSFET) such as complementary metal-insulator-semiconductor (CMOS) transistor, system large scale integration (LSI), and image sensors such as CMOS imaging sensors (CIS), micro-electro-mechanical systems (MEMS), active devices, passive devices, etc.

However, the semiconductor devices 220_TR of the active layer 220 are not limited thereto, and may include a semiconductor device having a three-dimensional structure such as a high bandwidth memory (HBM), a buried vertical NAND (BVNAND), a backside power delivery network (BSPDN), and a virtual synchronous DRAM (VSDRAM).

The wiring structure 220_WS may include a plurality of contacts 223 and a plurality of wiring layers 224. Each of the plurality of contacts 223 may be electrically connected to one of the source/drain regions 222. The plurality of wiring layers 224 may be electrically connected to the plurality of contacts 223. The plurality of wiring layers 224 may have a multilayer structure including a plurality of metal layers arranged at different vertical levels.

Referring to FIG. 3, the X-ray source layer XS may be formed on the first surface 200_1 of the semiconductor substrate 200. For example, the X-ray source layer XS may be spaced apart from the base layer 210 of the semiconductor substrate 200 with the active layer 220 of the semiconductor substrate 200 therebetween. However, example embodiments are not limited thereto, and the X-ray source layer XS may be formed on the second surface 200_2 of the semiconductor substrate 200.

For example, the X-ray source layer XS may cover the entirety of first surface 200_1 of the semiconductor substrate 200. For example, side surfaces of the X-ray source layer XS may be coplanar (or vertically flushed, or aligned) with side surfaces of the semiconductor substrate 200. In some example embodiments, the X-ray source layer XS may be in a range of 0.5 μm (or about 0.5 μm) to 2 μm (or about 2 μm). In some example embodiments, the X-ray source layer XS may include at least one of tungsten (W) and copper (Cu).

In some example embodiments, the X-ray source layer XS may be formed by performing a separate operation after forming the semiconductor substrate 200. In some example embodiments, the X-ray source layer XS may be formed by performing physical vapor deposition (PVD), chemical vapor deposition (CVD), and atomic layer deposition (ALD) on the first surface 200_1 of the semiconductor substrate 200. For example, the X-ray source layer XS may be conformally formed on the active layer 220, or alternatively, on the first surface 200_1 of the semiconductor substrate 200. For example, the X-ray source layer XS may have a flat plate-like shape.

In some example embodiments, the X-ray source layer XS may be formed during the process of manufacturing the semiconductor substrate 200. For example, the X-ray source layer XS may be a portion of the wiring structure 220_WS of the active layer 220 of the semiconductor substrate 200. For example, the X-ray source layer XS may be integrally formed with the wiring layer located at the top of the plurality of wiring layers 224 of the wiring structure 220_WS.

The X-ray source layer XS may be formed on the first surface 200_1 or the second surface 200_2 of the semiconductor substrate 200, and may be stationary or fixed on the semiconductor substrate 200. For example, since the X-ray source layer XS is fixed to the semiconductor substrate 200, it may be possible to limit a situation in which the X-ray source layer XS and the semiconductor substrate 200 collide with each other while the semiconductor substrate 200 is rotated or moved.

Referring to FIG. 3, together with FIGS. 1 and 2, the semiconductor substrate 200 on which the X-ray source layer XS is formed on the first surface 200_1 may be mounted in the semiconductor device imaging apparatus 100. In some example embodiments, the semiconductor substrate 200 may be mounted using an assembly that secures the semiconductor substrate 200 and that can be rotated or otherwise moved in desired directions.

The semiconductor device imaging apparatus 100 may include an electron-beam feeder 110, an anode 120, a magnetic lens 130, a scanning coil 140, an objective lens 150, and/or an X-ray detector 160.

The electron-beam feeder 110 may use, for example, a Schottky type or a thermoelectric emission type electron gun. Electron-beams EB may be emitted by applying an acceleration voltage to the electron-beam feeder 110. The anode 120 is an acceleration electrode, and the electron-beams EB are accelerated by a voltage applied between the electron-beam feeder 110 and the anode 120.

The magnetic lens 130 may function to focus and accelerate the electron-beams EB. The scanning coil 140 may scan the electron-beams EB on a specimen, for example, the semiconductor substrate 200 in one dimension or two dimensions. For example, the scanning directions of the electron-beams EB may be changed according to the frequency applied to the scanning coil 140. The objective lens 150 may be positioned between the semiconductor substrate 200 and the scanning coil 140 to focus the electron-beams EB deflected by the scanning coil 140 on the X-ray source layer XS formed on the first surface 200_1 of the semiconductor substrate 200.

The X-ray detector 160 may be arranged to be spaced apart from the electron-beam feeder 110 in a first direction D1. For example, the X-ray detector 160 may be spaced apart from the electron-beam feeder 110 with a support on which the semiconductor substrate 200 is mounted therebetween. The X-ray detector 160 may detect X-rays XR that have passed through the semiconductor substrate 200.

The semiconductor substrate 200 may be mounted in the semiconductor device imaging apparatus 100 such that the X-ray source layer XS faces the electron-beam feeder 110. For example, the semiconductor substrate 200 may be positioned between the electron-beam feeder 110 and the X-ray detector 160. The first surface 200_1 of the semiconductor substrate 200 may face the electron-beam feeder 110, and the second surface 200_2 of the semiconductor substrate 200 may face the X-ray detector 160.

For example, a normal direction of the first surface 200_1 of the semiconductor substrate 200 may be parallel to the first direction D1. For example, the semiconductor substrate 200 may be mounted in the semiconductor device imaging apparatus 100 such that the first surface 200_1 of the semiconductor substrate 200 is perpendicular to a separation direction between the electron-beam feeder 110 and the X-ray detector 160. For example, the first surface 200_1 of the semiconductor substrate 200 and the top surface of the X-ray detector 160 may be parallel to each other.

Next, the electron-beam feeder 110 may irradiate electron-beams EB toward the X-ray source layer XS. For example, the electron-beams EB may pass through the anode 120, the magnetic lens 130, the scanning coil 140, and the objective lens 150 to reach the X-ray source layer XS. For example, the electron-beam EB irradiated or emitted by the electron-beam feeder 110 may be directly incident on the X-ray source layer XS formed on the semiconductor substrate 200. In some example embodiments, the semiconductor device imaging apparatus 100 may not include equipment for separately generating X-rays XR. For example, the semiconductor device imaging method S100 may form the X-ray XR through the X-ray source layer XS formed on the semiconductor substrate 200, and thus, the semiconductor device imaging apparatus 100 itself may not include an X-ray source layer.

The electron-beams EB may be directly incident on the X-ray source layer XS, and atoms of the X-ray source layer XS may form X-rays XR by interaction with the electron-beams EB. The X-rays XR, formed by the X-ray source layer XS, may penetrate the semiconductor substrate 200 and may be emitted from the second surface 200_2 of the semiconductor substrate 200.

Since the X-ray source layer XS is formed on the first surface 200_1 of the semiconductor substrate 200, the X-rays XR formed by the X-ray source layer XS may be directly transferred from the X-ray source layer XS to the semiconductor substrate 200. For example, since the X-ray source layer XS and the semiconductor substrate 200 are in contact (e.g., direct contact) with each other, the X-rays XR formed in the X-ray source layer XS may pass through the X-ray source layer XS and directly enter the semiconductor substrate 200 without passing through a separate medium.

The X-rays XR emitted from the second surface 200_2 of the semiconductor substrate 200 may be incident on the X-ray detector 160 facing the second surface 200_2 of the semiconductor substrate 200. In some example embodiments, when the semiconductor substrate 200 is photographed by adjusting the region in which the electron-beams EB hit the X-ray source layer XS, a two-dimensional (2D) X-ray image of the active layer 220 of the semiconductor substrate 200 may be obtained by the X-ray detector 160. One or more of the X-ray images obtained by the X-ray detector 160 may be analyzed or further processed to determine an interior structure of the semiconductor substrate 200 with relatively higher precision. By obtaining the interior structure of the semiconductor substrate 200, any defects or design variations in the semiconductor substrate 200, for example, in the semiconductor device 220_TR and/or the wiring structure 220_WS may be determined.

In some example embodiments, the semiconductor substrate 200 may be mounted in the semiconductor device imaging apparatus 100 such that a normal direction of the first surface 200_1 of the semiconductor substrate 200 is not parallel (or intersecting) to the first direction D1, and a three-dimensional (3D) X-ray image of the active layer 220 of the semiconductor substrate 200 may be obtained by repeating the photographing process.

In X-ray imaging, the magnification may vary depending on the distance between the X-ray source layer XS forming the X-rays XR, the semiconductor substrate 200 to be measured by the X-rays XR, and the X-ray detector 160 detecting the X-rays XR. For example, as the distance between the X-ray source layer XS and the semiconductor substrate 200 is relatively smaller, the magnification of the X-ray image may be relatively higher. As a result, a relatively higher-resolution X-ray image may be obtained.

For example, the magnification of the X-ray image may have a value of M that satisfies Equation 1 below.

$$M = \frac{{Z}_{1} + {Z}_{2}}{{Z}_{1}}$$

### <Equation 1>

In this case, Z₁ may be a distance between the X-ray source layer XS and the semiconductor substrate 200, and Z₂ may be a distance between the semiconductor substrate 200 and the X-ray detector 160. (Z₁+Z₂) may be a distance from the X-ray source layer XS to the X-ray detector 160.

For example, Z₁ may be a distance from a place where the X-rays XR are formed in the X-ray source layer XS, which may be a place where the electron-beams EB strikes, to the semiconductor device 220_TR in the semiconductor substrate 200. Z₂ may be a distance from the semiconductor device 220_TR of the semiconductor substrate 200 to the X-ray detector 160.

In the semiconductor device imaging method, according to some example embodiments, Z₁ may be substantially the same as the thickness of the X-ray source layer XS. The magnification of the X-ray image may be adjusted by adjusting the thickness of the X-ray source layer XS. For example, the magnification of the X-ray image may be adjusted by adjusting the distance between the X-ray source layer XS and the X-ray detector 160.

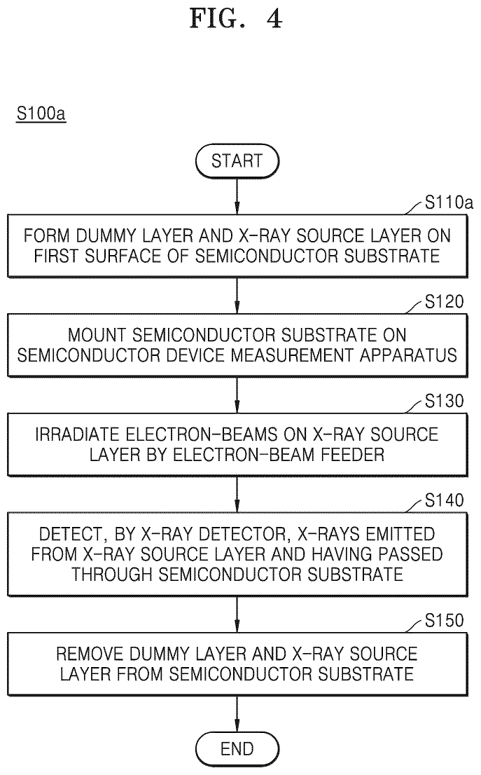

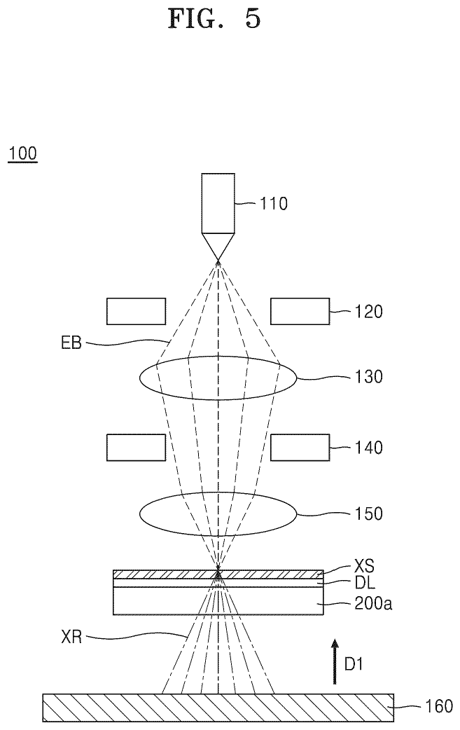

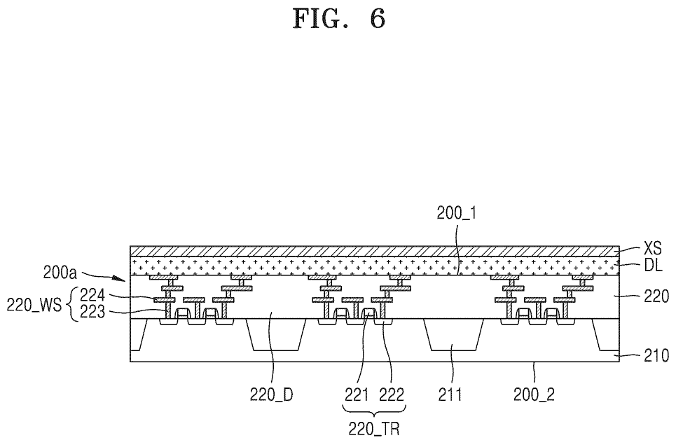

FIG. 4 is a flowchart schematically illustrating a method of imaging a semiconductor device (S100a) according to some example embodiments. FIG. 5 is a diagram schematically illustrating a semiconductor device imaging apparatus 100 and a semiconductor substrate 200a according to some example embodiments. FIG. 6 is an enlarged view of a portion of the semiconductor substrate 200a of FIG. 5. It is understood that additional operations can be provided before, during, and after the operations in FIG. 4, and some of the operations described below can be replaced or eliminated, for additional embodiments of the method. The order of the operations/processes may be interchangeable, or two or more operations can be performed simultaneously.

Referring to FIGS. 4 to 6, a semiconductor device imaging method S100a may include an operation S110a of forming an X-ray source layer XS and a dummy layer DL on a first surface 200_1 of a semiconductor substrate 200a, an operation S120 of mounting the semiconductor substrate 200a on a semiconductor device imaging apparatus 100, an operation S130 of irradiating electron-beams EB on the X-ray source layer XS by an electron-beam feeder 110, and an operation S140 of detecting, by an X-ray detector 160, X-rays XR emitted from the X-ray source layer XS and having passed through the semiconductor substrate 200a.

In some example embodiments, the semiconductor device imaging method S100a may further include an operation S150 of removing the dummy layer DL and the X-ray source layer XS from the semiconductor substrate 200a. For example, the semiconductor device imaging method S100a may measure the semiconductor device 220_TR of the semiconductor substrate 200a in a non-destructive manner.

The semiconductor device imaging method S100a described below may be same as or similar in some respects to the semiconductor device imaging method S100 described above with reference to FIG. 1, and may be best understood with reference thereto. In some example embodiments, the semiconductor device imaging method S100a of FIG. 4 further includes an operation of forming the dummy layer DL in the semiconductor device imaging method S100 of FIG. 1.

The dummy layer DL and the X-ray source layer XS may be formed on one surface of the semiconductor substrate 200a. The dummy layer DL may be arranged between the semiconductor substrate 200a and the X-ray source layer XS. The X-ray source layer XS may be spaced apart from the semiconductor substrate 200a with the dummy layer DL therebetween.

The semiconductor substrate 200a may include a base layer 210 and an active layer 220. In some example embodiments, the active layer 220 of the semiconductor substrate 200a may include a semiconductor device 220_TR and a wiring structure 220_WS. For example, a surface on which the active layer 220 is formed among the upper and lower surfaces of the semiconductor substrate 200a may be referred to as a first surface 200_1, and a surface on which the base layer 210 is located among the upper and lower surfaces of the semiconductor substrate 200a may be referred to as a second surface 200_2. The dummy layer DL and the X-ray source layer XS may be formed on the first surface 200_1 of the semiconductor substrate 200a. However, example embodiments are not limited thereto, and the dummy layer DL and the X-ray source layer XS may be formed on the second surface 200_2 of the semiconductor substrate 200a.

In some example embodiments, the dummy layer DL may not be electrically connected to the semiconductor substrate 200a. In some example embodiments, the dummy layer DL may cover the entirety of the first surface 200_1 of the semiconductor substrate 200a. In some example embodiments, the dummy layer DL may include silicon oxide, silicon nitride, or the like.

In some example embodiments, the dummy layer DL may be formed by performing a separate operation after forming the semiconductor substrate 200a. In some example embodiments, the dummy layer DL may be formed by performing physical vapor deposition (PVD), chemical vapor deposition (CVD), and atomic layer deposition (ALD) on the first surface 200_1 of the semiconductor substrate 200a.

However, example embodiments are not limited thereto, and the dummy layer DL may be formed during the process of forming the semiconductor substrate 200a. For example, the dummy layer DL may be a portion of an insulating layer 220_D of the active layer 220 of the semiconductor substrate 200a. For example, a portion of the insulating layer 220_D positioned at the uppermost end may be referred to as a dummy layer DL.

In some example embodiments, the X-ray source layer XS may be positioned on a surface of the dummy layer DL opposite to a surface thereof in contact with the semiconductor substrate 200a. For example, after the dummy layer DL is formed, an X-ray source layer XS may be formed on the dummy layer DL. The X-ray source layer XS may be spaced apart from the semiconductor substrate 200a and may not be electrically connected to the semiconductor substrate 200a. The X-ray source layer XS may completely cover one surface of the dummy layer DL.

In some example embodiments, a thickness of the dummy layer DL may be different from a thickness of the X-ray source layer XS. In some example embodiments, a thickness of the dummy layer DL may be greater than a thickness of the X-ray source layer XS. For example, the thickness of the dummy layer DL may be in a range of 5 μm (or about 5 μm) to 15 μm (or about 15 μm).

Next, the semiconductor substrate 200a may be mounted in the semiconductor device imaging apparatus 100 such that the first surface 200_1 of the semiconductor substrate 200a faces the electron-beam feeder 110. For example, the semiconductor substrate 200a may be positioned such that the dummy layer DL and the X-ray source layer XS formed on the semiconductor substrate 200a face the electron-beam feeder 110.

The electron-beam feeder 110 and the X-ray detector 160 of the semiconductor device imaging apparatus 100 may be spaced apart from each other in the first direction D1. The semiconductor substrate 200a may be positioned between the electron-beam feeder 110 and the X-ray detector 160. For example, the X-ray source layer XS may face the electron-beam feeder 110, and the base layer 210 of the semiconductor substrate 200a may face the X-ray detector 160.

In some example embodiments, the semiconductor substrate 200a may be mounted in the semiconductor device imaging apparatus 100 such that a normal direction of the first surface 200_1 of the semiconductor substrate 200a is parallel to the first direction D1. However, example embodiments are not limited thereto, and the semiconductor substrate 200a may be mounted inclined with respect to the semiconductor device imaging apparatus 100 such that a normal direction of the first surface 200_1 of the semiconductor substrate 200a is angled or skewed with reference to the first direction D1 at an angle other than 90°. In other words, a normal direction of the first surface 200_1 of the semiconductor substrate 200a may not be orthogonal to the first direction D1.

The electron-beam feeder 110 may emit electron-beams EB toward the X-ray source layer XS. The electron-beams EB and the X-ray source layer XS may interact to form X-rays XR. The X-rays XR may pass through the dummy layer DL and the semiconductor substrate 200a and may be incident on the X-ray detector 160. The X-rays XR formed in the X-ray source layer XS may pass through the dummy layer DL to reach the semiconductor substrate 200a. For example, the distance between the X-ray source layer XS and the semiconductor substrate 200a may be substantially the same as the thickness of the dummy layer DL.

In the semiconductor device imaging method S100a, the distance between the X-ray source layer XS and the semiconductor device 220_TR of the semiconductor substrate 200a may be adjusted through the dummy layer DL. For example, the magnification of the X-ray image may be adjusted by adjusting the thickness of the dummy layer DL.

After X-ray imaging is completed, the dummy layer DL and the X-ray source layer XS may be removed from the semiconductor substrate 200a. For example, the peelability of the dummy layer DL and the semiconductor substrate 200a may be higher than that of the X-ray source layer XS and the semiconductor substrate 200a. For example, the dummy layer DL may be removed from the semiconductor substrate 200a by applying heat to the dummy layer DL.

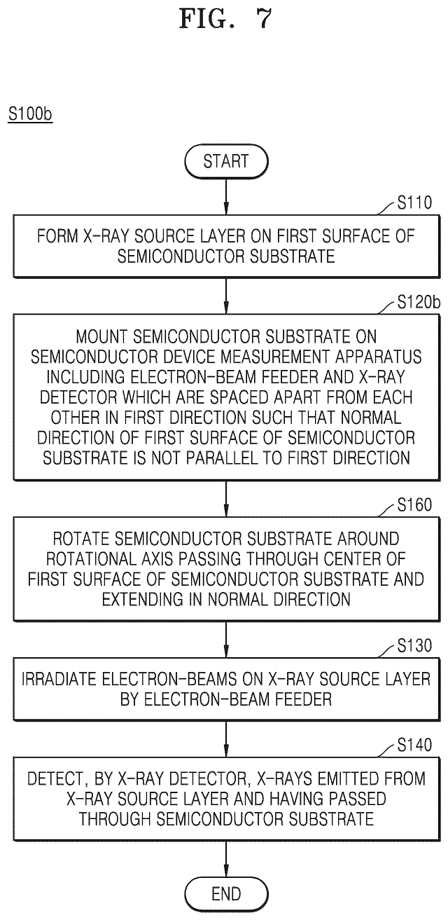

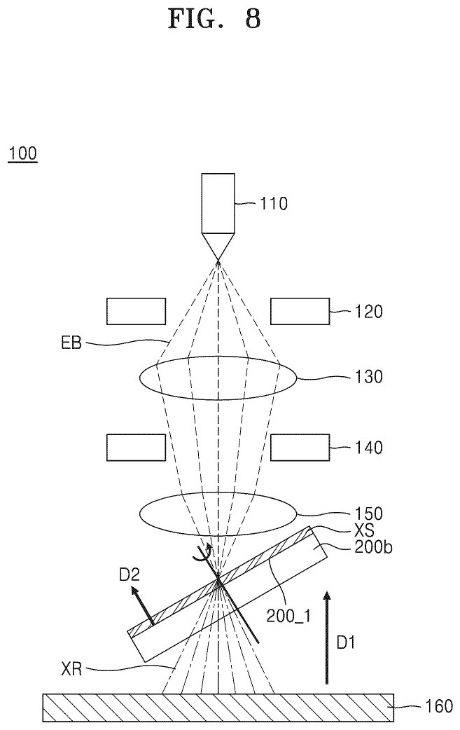

FIG. 7 is a flowchart schematically illustrating a method of imaging a semiconductor device (S100b) according to some example embodiments. FIG. 8 is a diagram schematically illustrating a semiconductor device imaging apparatus 100 and a semiconductor substrate 200b according to some example embodiments. It is understood that additional operations can be provided before, during, and after the operations in FIG. 7, and some of the operations described below can be replaced or eliminated, for additional embodiments of the method. The order of the operations/processes may be interchangeable, or two or more operations can be performed simultaneously.

Referring to FIGS. 7 and 8, a semiconductor device imaging method S100b includes an operation S110 of forming an X-ray source layer XS on a first surface 200_1 of a semiconductor substrate 200b, an operation S120b of mounting the semiconductor substrate 200b on a semiconductor device imaging apparatus 100, an operation S160 of rotating the semiconductor substrate 200b, an operation S130 of irradiating electron-beams EB onto the X-ray source layer XS by an electron-beam feeder 110, and an operation S140 of detecting, by an X-ray detector 160, X-rays XR emitted from the X-ray source layer XS and having passed through the semiconductor substrate 200b.

The semiconductor device imaging method S100b described below may be same as or similar in some respects to the semiconductor device imaging method S100 of FIG. 1, and may be best understood with reference thereto.

The X-ray source layer XS may be formed on one surface of the semiconductor substrate 200b including the semiconductor device 220_TR (see FIG. 3). In some example embodiments, the operation of forming the X-ray source layer XS may be performed as a separate process after the fabrication of the semiconductor substrate 200b is completed. In addition or alternatively, the X-ray source layer XS may be formed together in the process of manufacturing the active layer 220 (see FIG. 3) of the semiconductor substrate 200b.

In some example embodiments, the X-ray source layer XS may be formed on the first surface 200_1 of the semiconductor substrate 200b. For example, by forming the X-ray source layer XS such that the X-ray source layer XS contacts the active layer of the semiconductor substrate 200b, the distance between the X-ray source layer XS and the semiconductor device to be measured may be reduced.

Thereafter, the semiconductor substrate 200b may be mounted in the semiconductor device imaging apparatus 100. For example, the semiconductor substrate 200b may be mounted in the semiconductor device imaging apparatus 100 such that the X-ray source layer XS faces the electron-beam feeder 110 of the semiconductor device imaging apparatus 100. Accordingly, the electron-beams EB emitted by the electron-beam feeder 110 may be directly incident on the X-ray source layer XS.

The electron-beam feeder 110 and the X-ray detector 160 of the semiconductor device imaging apparatus 100 may be spaced apart from each other in the first direction D1. The semiconductor substrate 200b may be mounted in the semiconductor device imaging apparatus 100 between the electron-beam feeder 110 and the X-ray detector 160 such that the X-ray source layer XS faces the electron-beam feeder 110.

In some example embodiments, and as illustrated in FIG. 8, when a normal direction of the first surface 200_1 of the semiconductor substrate 200b is the second direction D2, the first direction D1 and the second direction D2 may not be parallel (e.g., may be intersecting) to each other. For example, the first direction D1 and the second direction D2 may intersect with each other. For example, when the electron-beam feeder 110 and the X-ray detector 160 are spaced apart in the vertical direction, the semiconductor substrate 200b may be mounted in the semiconductor device imaging apparatus 100 while being inclined.

After mounting the semiconductor substrate 200b on the semiconductor device imaging apparatus 100, the semiconductor substrate 200b may be rotated. The semiconductor substrate 200b may be rotated (e.g., clockwise or counter-clockwise) about a rotational axis extending in the second direction D2 while passing through the center of the first surface 200_1 of the semiconductor substrate 200b. For example, as the inclined semiconductor substrate 200b is rotated about the rotational axis, a semiconductor device 220_TR (see FIG. 3) may be measured in various directions. Accordingly, even in the case of a semiconductor device having a three-dimensional structure, the semiconductor device imaging method S100b may observe or otherwise determine the shape of the semiconductor device with relatively accuracy.

The electron-beam feeder 110 may emit electron-beams EB toward the X-ray source layer XS. For example, the electron-beam feeder 110 may emit the electron-beams EB toward the X-ray source layer XS while the semiconductor substrate 200b is rotated. Accordingly, depending on the rotation angle of the semiconductor substrate 200b, the portion of the semiconductor substrate 200b through which the X-rays XR formed in the X-ray source layer XS by the electron-beams EB pass may vary.

The X-rays XR penetrate a portion of the semiconductor substrate 200b and is incident on the X-ray detector 160, such that an X-ray image may be produced. By synthesizing or processing the X-ray images, the interior structure of the semiconductor substrate 200b may be observed with relatively higher precision.

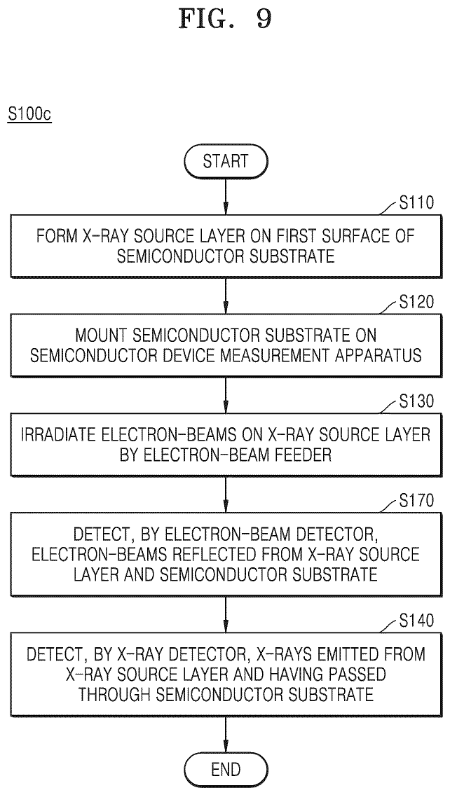

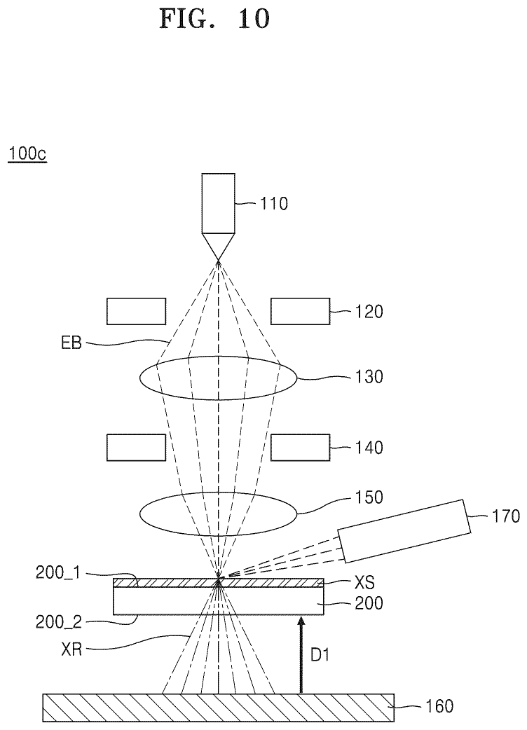

FIG. 9 is a flowchart schematically illustrating a method of imaging a semiconductor device (S100c) according to some example embodiments. FIG. 10 is a diagram schematically illustrating a semiconductor device imaging apparatus 100c and a semiconductor substrate 200 according to some example embodiments. It is understood that additional operations can be provided before, during, and after the operations in FIG. 9, and some of the operations described below can be replaced or eliminated, for additional embodiments of the method. The order of the operations/processes may be interchangeable, or two or more operations can be performed simultaneously.

Referring to FIGS. 9 and 10, a semiconductor device imaging method S100c includes an operation S110 of forming an X-ray source layer XS on a first surface 200_1 of a semiconductor substrate 200, an operation S120 of mounting the semiconductor substrate 200 on a semiconductor device imaging apparatus 100, an operation S130 of irradiating electron-beams EB onto the X-ray source layer XS by an electron-beam feeder 110, an operation S170 of detecting, by an electron-beam detector 170, the electron-beams EB reflected from the X-ray source layer XS and the semiconductor substrate 200, and an operation S140 of detecting the X-rays XR emitted from the X-ray source layer XS and having passed through the semiconductor substrate 200b.

The semiconductor device imaging method S100c described below may be same as or similar in some respects to the semiconductor device imaging method S100 of FIG. 1, and may be best understood with reference thereto.

The X-ray source layer XS may be formed on one surface of the semiconductor substrate 200 including the semiconductor device 220_TR (see FIG. 3). In some example embodiments, the operation of forming the X-ray source layer XS may be performed as a separate process after the fabrication of the semiconductor substrate 200 is completed. In addition, the X-ray source layer XS may be formed together in the process of manufacturing the active layer 220 (see FIG. 3) of the semiconductor substrate 200.

In some example embodiments, when the X-ray source layer XS is formed in the process of forming the semiconductor substrate 200, the operation S110 of forming the X-ray source layer XS on the semiconductor substrate 200 may be omitted.

The semiconductor substrate 200 on which the X-ray source layer XS is formed on the first surface 200_1 may be mounted in the semiconductor device imaging apparatus 100c. The semiconductor device imaging apparatus 100c may include an electron-beam feeder 110, an anode 120, a magnetic lens 130, a scanning coil 140, an objective lens 150, an X-ray detector 160, and an electron-beam detector 170.

The electron-beam feeder 110 may be spaced apart from the X-ray detector 160 in the first direction D1. The electron-beam detector 170 may be spaced apart from the electron-beam feeder 110 in the first direction D1. For example, the electron-beam detector 170 may be positioned between the electron-beam feeder 110 and the X-ray detector 160.

The semiconductor substrate 200 may be positioned between the electron-beam feeder 110 and the X-ray detector 160. The first surface 200_1 of the semiconductor substrate 200 may face the electron-beam feeder 110 and the electron-beam detector 170, and the second surface 200_2 of the semiconductor substrate 200 may face the X-ray detector 160. For example, the semiconductor substrate 200 may be mounted in the semiconductor device imaging apparatus 100c such that the electron-beam feeder 110 and the electron-beam detector 170 are located above the first surface 200_1 of the semiconductor substrate 200 and the X-ray detector 160 is located below the second surface 200_2 of the semiconductor substrate 200.

For example, the electron-beam feeder 110 and the X-ray detector 160 may be spaced apart from each other with the semiconductor substrate 200 therebetween. The electron-beam detector 170 and the X-ray detector 160 may be spaced apart from each other with the semiconductor substrate 200 therebetween.

The electron-beam feeder 110 may irradiate electron-beams EB toward the X-ray source layer XS. The electron-beams EB emitted by the electron-beam feeder 110 may be directly incident on the X-ray source layer XS formed on the semiconductor substrate 200.

Some portions of the electron-beams EB reaching the X-ray source layer XS may interact with the X-ray source layer XS, and some other portions of the electron-beams EB reaching the X-ray source layer XS may be reflected from the X-ray source layer XS and/or the first surface 200_1 of the semiconductor substrate 200. The X-ray source layer XS may interact with the electron-beams EB to form X-rays XR.

The X-rays XR formed by the X-ray source layer XS may penetrate the semiconductor substrate 200 and may be incident on the X-ray detector 160. The electron-beams EB reflected from the X-ray source layer XS and/or the electron-beams EB reflected from the semiconductor substrate 200 may be incident on the electron-beam detector 170.

In some example embodiments, the electron-beam detector 170 may detect secondary electrons generated in the X-ray source layer XS and the semiconductor substrate 200 by the electron-beams EB. Through the electron-beam detector 170, an image of the shape and pattern of the semiconductor device 220_TR (refer to FIG. 3) formed on the semiconductor substrate 200 may be generated. The image of the shape and pattern of the semiconductor device 220_TR may be further analyzed or processed to determine any defects or design deviations present in the semiconductor device 220_TR.

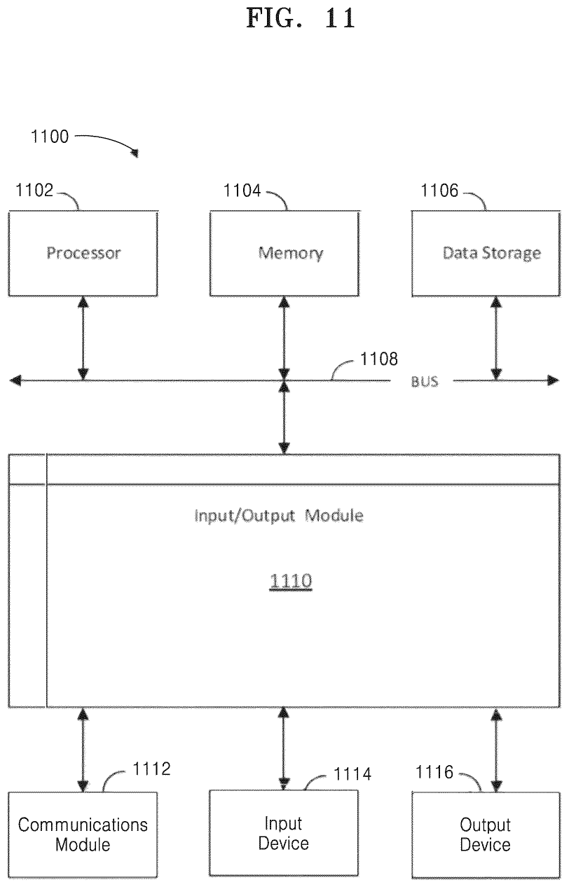

FIG. 11 is a block diagram illustrating an example computer system 1100 with which the methods and operations illustrated in FIGS. 1, 4, 7, and 9, and other tasks described herein can be implemented, according to some example embodiments. According to some example embodiments, the computer system 1100 may analyze or process the images obtained by the X-ray detector 160. In some example embodiments, computer system 1100 may be implemented using hardware or a combination of software and hardware, either in a dedicated server, integrated into another entity, or distributed across multiple entities.

Computer system 1100 includes a bus 1108 or other communication mechanism for communicating information, and a processor 1102 coupled with bus 1108 for processing information. By way of example, computer system 1100 can be implemented with one or more processors 1102. Processor 1102 can be a microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1100 includes, in addition to hardware, code that creates an execution environment for the computer program in question, e.g., code that constitutes processor firmware, a protocol stack, a database management system, an operating system, or a combination of one or more of them stored in an included memory 1104, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 1108 for storing information and instructions to be executed by processor 1102. Processor 1102 and memory 1104 can be supplemented by, or incorporated in, special purpose logic circuitry.

The memory 1104 may store an instruction program, and the processor 1102 may perform a function (e.g., methods and operations illustrated in FIGS. 1, 4, 7, and 9) by executing the stored instruction program.

The instructions may be stored in memory 1104 and implemented in one or more computer program products, e.g., one or more modules of computer program instructions encoded on a computer readable medium for execution by, or to control the operation of, the computer system 1100. The instructions may include a computer program, a code, or any combination thereof, and may transform the processor 1102 for a special purpose by instructing and/or configuring the processor independently or collectively to operate as desired. Memory 1104 may also be used for storing temporary variable or other intermediate information during execution of instructions to be executed by processor 1102.

Computer system 1100 further includes a data storage device 1106 such as a magnetic disk or optical disk, coupled to bus 1108 for storing information and instructions.

Computer system 1100 is coupled via input/output module 1110 to various devices. The input/output module 1110 is any input/output module. Example input/output modules 1110 include data ports such as USB ports. The input/output module 1110 is configured to connect to a communications module 1112. Example communications modules 1112 include networking interface cards, such as Ethernet cards and modems. In certain aspects, the input/output module 1110 is configured to connect to a plurality of devices, such as an input device 1114 and/or an output device 1116. Example input devices 1114 include a keyboard and a pointing device, e.g., a mouse or a trackball, by which a user can provide input to the computer system 1100. Example output devices 1116 include display devices, such as a LED (light emitting diode), CRT (cathode ray tube), or LCD (liquid crystal display) screen, for displaying information to the user.

Methods as disclosed herein may be performed by computer system 1100 in response to processor 1102 executing one or more sequences of one or more instructions contained in memory 1104. Such instructions may be read into memory 1104 from another machine-readable medium, such as data storage device 1106. Execution of the sequences of instructions contained in memory 1104 causes processor 1102 to perform the operations and other tasks described herein. One or more processors in a multi-processing arrangement may also be employed to execute the sequences of instructions contained in memory 1104. In alternative aspects, hard-wired circuitry may be used in place of or in combination with software instructions to implement various aspects of the present disclosure. Thus, aspects of the present disclosure are not limited to any specific combination of hardware circuitry and software.

Computer system 1100 includes servers and personal computer devices. A personal computing device and server are generally remote from each other and typically interact through a communication network. The relationship of client and server arises by virtue of computer programs running on the respective computers and having a client-server relationship to each other. Computer system 1100 can be, for example, and without limitation, a desktop computer, laptop computer, or tablet computer.

The term “machine-readable storage medium” or “computer readable medium” as used herein refers to any medium or media that participates in providing instructions or data to processor 1102 for execution. Such a medium may take many forms, including, but not limited to, non-volatile media, volatile media, and transmission media. Non-volatile media include, for example, optical disks, magnetic disks, or flash memory, such as data storage device 1106. Volatile media include dynamic memory, such as memory 1104. Transmission media include coaxial cables, copper wire, and fiber optics, including the wires that comprise bus 1108. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As described herein, any devices, systems, modules, portions, units, controllers, circuits, and/or portions thereof according to any of the example embodiments, and/or any portions thereof (including, without limitation, the electron-beam feeder 110, the X-ray detector 160, the electron-beam detector 170, any portion thereof, or the like) may include, may be included in, and/or may be implemented by one or more instances of processing circuitry such as hardware including logic circuits; a hardware/software combination such as a processor executing software; or a combination thereof. For example, the processing circuitry more specifically may include, but is not limited to, a central processing unit (CPU), an arithmetic logic unit (ALU), a graphics processing unit (GPU), an application processor (AP), a digital signal processor (DSP), a microcomputer, a field programmable gate array (FPGA), and programmable logic unit, a microprocessor, application-specific integrated circuit (ASIC), a neural network processing unit (NPU), an Electronic Control Unit (ECU), an Image Signal Processor (ISP), and the like. In some example embodiments, the processing circuitry may include a non-transitory computer readable storage device (e.g., a memory), for example a solid state drive (SSD), storing a program of instructions, and a processor (e.g., CPU) configured to execute the program of instructions to implement the functionality and/or methods performed by some or all of any devices, systems, modules, portions, units, controllers, circuits, and/or portions thereof according to any of the example embodiments.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

What is claimed is:

1. A method of imaging a semiconductor device, the method comprising:
forming an X-ray source layer on a first surface of a semiconductor substrate;
mounting the semiconductor substrate in a semiconductor device imaging apparatus, the semiconductor device imaging apparatus including an electron-beam feeder and an X-ray detector;
irradiating electron-beams on the X-ray source layer of the semiconductor substrate using the electron-beam feeder; and
measuring X-rays emitted from the X-ray source layer and having passed through the semiconductor substrate using the X-ray detector.

2. The method of claim 1, wherein forming the X-ray source layer includes forming the X-ray source layer to cover an entirely of the first surface of the semiconductor substrate.

3. The method of claim 1, wherein the first surface of the semiconductor substrate includes an active layer, and at least one semiconductor device is formed on the active layer.

4. The method of claim 1, wherein a thickness of the X-ray source layer is in a range of 0.5 μm to 2 μm.

5. The method of claim 1, wherein the electron-beams irradiated by the electron-beam feeder are directly incident on the X-ray source layer formed on the semiconductor substrate.

6. The method of claim 1, wherein
the electron-beam feeder and the X-ray detector are spaced apart in a first direction, and
the method further includes,
mounting the semiconductor substrate in the semiconductor device imaging apparatus such that the first surface of the semiconductor substrate faces the electron-beam feeder and a second surface of the semiconductor substrate faces the X-ray detector, the second surface being opposite to the first surface.

7. The method of claim 6, wherein mounting the semiconductor substrate includes mounting the semiconductor substrate in the semiconductor device imaging apparatus such that a line that is normal to the first surface of the semiconductor substrate is in a second direction, and wherein the second direction and the first direction intersect each other.

8. The method of claim 7, further comprising rotating the semiconductor substrate about a rotational axis that passes through a center of the first surface of the semiconductor substrate and extends in the second direction.

9. The method of claim 1, further comprising, before the forming of the X-ray source layer, forming a dummy layer on the first surface of the semiconductor substrate.

10. The method of claim 9, wherein a thickness of the dummy layer is in a range of 5 μm to 15 μm.

11. The method of claim 9, further comprising, after the measuring the X-rays, removing the X-ray source layer and the dummy layer from the semiconductor substrate.

12. The method of claim 1, wherein the X-ray source layer includes at least one of tungsten or copper.

13. The method of claim 1, wherein
the semiconductor device imaging apparatus further includes an electron-beam detector, and the method further comprises,
measuring, using the electron-beam detector, electron-beams reflected from at least one of the X-ray source layer or the semiconductor substrate among the electron-beams irradiated to the X-ray source layer.

14. The method of claim 13, wherein mounting the semiconductor substrate in the semiconductor device imaging apparatus includes mounting the semiconductor substrate such that the first surface of the semiconductor substrate faces the electron-beam feeder and the electron-beam detector, and a second surface of the semiconductor substrate faces the X-ray detector, the second surface being opposite to the first surface.

15. A method of imaging a semiconductor device, the method comprising:
mounting a semiconductor substrate in a semiconductor device imaging apparatus including an electron-beam feeder and an X-ray detector;
irradiating electron-beams on an X-ray source layer of the semiconductor substrate using the electron-beam feeder; and
measuring X-rays emitted from the X-ray source layer and having passed through the semiconductor substrate using the X-ray detector, wherein
the semiconductor substrate includes the X-ray source layer on a first surface of the semiconductor substrate, and the semiconductor substrate is mounted in the semiconductor device imaging apparatus such that the X-ray source layer faces the electron-beam feeder.

16. The method of claim 15, wherein the semiconductor substrate further includes a dummy layer arranged between the first surface of the semiconductor substrate and the X-ray source layer.

17. The method of claim 15, wherein irradiating the electron-beams on the X-ray source layer includes irradiating the electron-beams directly on the X-ray source layer of the semiconductor substrate.

18. The method of claim 15, further comprising rotating the semiconductor substrate about a rotational axis that passes through a center of the first surface of the semiconductor substrate and extending in a direction of a line that is normal to the first surface of the semiconductor substrate.

19. A method of imaging a semiconductor device, the method comprising:
forming a dummy layer and an X-ray source layer on a first surface of a semiconductor substrate, the dummy layer and the X-ray source layer covering an entirety of the first surface;
mounting the semiconductor substrate between an electron-beam feeder and an X-ray detector of a semiconductor device imaging apparatus, the semiconductor substrate being mounted such that the X-ray source layer faces the electron-beam feeder, and the electron-beam feeder and the X-ray detector being spaced apart in a first direction from each other;
rotating the semiconductor substrate about a rotational axis passing through a center of the first surface of the semiconductor substrate and extending in a direction of a line that is normal to the first surface of the semiconductor substrate;
irradiating electron-beams toward the X-ray source layer of the semiconductor substrate using the electron-beam feeder;
directly striking the X-ray source layer with the electron-beams to cause the X-ray source layer to form X-rays; and
detecting the X-rays that pass through the semiconductor substrate using the X-ray detector, wherein,
in the mounting of the semiconductor substrate, the semiconductor substrate is mounted in the semiconductor device imaging apparatus such that the direction of the line normal to the first surface of the semiconductor substrate and the first direction intersect each other.

20. The method of claim 19, wherein forming the dummy layer and the X-ray source layer includes,
forming the dummy layer on the first surface of the semiconductor substrate, and
forming the X-ray source layer on the dummy layer and having a thickness smaller than the dummy layer, and the method further includes removing the dummy layer and the X-ray source layer from the semiconductor substrate.
